SEQUENCE LISTING (5 sequences)

SEQ ID NO: 1 - protein, 27 aa, Artificial Sequence
  Protein Transduction Domain
PKKKRKVGLFGAIAGFIENGWEGMIDG

SEQ ID NO: 2 - DNA, 34 bp, Artificial
  CSK-F primer
gcaggctccaccatgtcagcaatacaggccgcct

SEQ ID NO: 3 - DNA, 37 bp, Artificial
  CSK-R primer
caagaaagctgggtgcaggtgcagctcgtgggttttg

SEQ ID NO: 4 - DNA, 37 bp, Artificial
  attB1-F2 primer
ggggacaagtttgtacaaaaaagcaggctccaccatg

SEQ ID NO: 5 - DNA, 30 bp, Artificial
  attB2-R2 primer
ggggaccactttgtacaagaaagctgggtg

(19) United States
(12) Patent Application Publication
(10) Pub. No.: US 2011/0217727 A1
(43) Pub. Date: Sep. 8, 2011

(54) METHOD FOR PATTERNING MAGNETIC MATERIALS IN LIVE CELL, METHOD FOR IMAGING PATTERN OF MAGNETIC MATERIALS, AND APPARATUS USED FOR SAME

(75) Inventor: Dae Joong Kim, Daejeon (KR)

(73) Assignee: MEDISCOV INC., Daejeon (KR)

(21) Appl. No.: 13/128,376

(22) PCT Filed: Nov. 9, 2009
(86) PCT No.: PCT/KR09/06541

§ 371 Date: May 9, 2011

(30) Foreign Application Priority Data
Nov. 10, 2008 (KR) 10-2008-0111326

(51) Int. Cl. C12Q 1/02; C12M 1/34; B82Y 15/00

(52) U.S. Cl. 435/29; 435/288.7; 977/838; 977/902

(57) ABSTRACT

The present invention provides a method for imaging a pattern of magnetic materials in a live cell comprising: preparing a plurality of magnetic materials magnetized in a direction of a line of magnetic force by applying a magnetic field, at least one of the magnetic materials being configured into a nanoparticle and the surface of at least one of the magnetic materials being modified; introducing a plurality of the magnetic materials into each live cell; providing the live cell with a label capable of binding to the magnetic material and imaging a pattern of the magnetic materials in a direction of a line of magnetic force; allowing a bundle of the lines of magnetic force to pass through the live cell in a direction by applying a focused magnetic field to the live cell; aligning a plurality of the magnetic materials in the live cell with the direction of the line of magnetic force by the applied magnetic field; and identifying an imaged pattern of the label capable of imaging the aligned pattern of the magnetic materials.

### TECHNICAL FIELD

The present invention relates to a method for patterning magnetic materials magnetized in a direction of a line of magnetic force in a live cell, a method for imaging a pattern of magnetic materials and an apparatus used for the same, more particularly to a method for patterning magnetic materials in a direction of a line of magnetic force in a live cell by applying a magnetic field, a method for imaging a pattern of magnetic materials in a direction of a line of magnetic force by a label and an apparatus used for the same.

### BACKGROUND ART

A cell is a basic structure and activity unit of an organism including a human being. A live cell is composed of cytoplasm and various subcellular organelles, such as nucleus, nucleolus, Golgi apparatus, endoplasmic reticulum, mitochondria, endosome, peroxisome, lysosome, and cytoskeleton, making it have such a complex structure.

The biological phenomena of live cells are regulated and maintained by various cellular components constituting the subcellular organelles or existing in the subcellular organelles, for example, macromolecules such as proteins, nucleic acids, polysaccharides and lipids, and small molecules such as amino acids, nucleotides, phosphoric acids, vitamins, amines and other organic compounds.

Cytoplasm of a cell is known as gel-like material characterized by viscoelasticity and thixotrophy (Luby-Phelps, et al. Probing the structure of cytoplasm. J. Cell Biol. (1986) 102, 2015-2022), and is known to have approximately four times higher fluid phase viscosity than water. In addition, cytoplasm has a barrier to limit free diffusion according to the size of macromolecules dissolved in the cytoplasm (Luby-Phelps, et al. Probing the structure of cytoplasm. J. Cell Biol. (1986) 102, 2015-2022).

The barrier of cell as stated above is assumed to be composed of filamentous meshwork and the average pore size of the filamentous meshwork is estimated to be 30-40 nm (Luby-Phelps, et al. Probing the structure of cytoplasm. J. Cell Biol. (1986) 102, 2015-2022).

Due to these characteristics of cytoplasm, long-chain polymers including oligomeric proteins, multi-enzyme complexes, mRNA, and etc., ribosome, and viruses are assumed to move very slowly or hardly and therefore it is reported that particles more than 25-30 nm can rarely move (Luby-Phelps, et al. Probing the structure of cytoplasm. J. Cell Biol. (1986) 102, 2015-2022; Arrio-Dupont, et al. Translational diffusion of globular proteins in the cytoplasm of cultured muscle cells. Biophys. J. (2000) 78, 901-907; Luby-Phelps, et al. Hindered diffusion of inert tracer particles in the cytoplasm of mouse 3T3 cells. Proc. Natl. Acad. Sci. USA (1987) 84, 4910-4913; Weiss, et al. Anomalous subdiffusion is a measure for cytoplasmic crowding in living cells. Biophys. J. (2004) 87, 3518-3524).

Traditionally, to study the structure of the cell and the function of cellular materials, a variety of methods have been used in biology, and especially, to study characteristics and structure of cytoplasm where various biological phenomena occur, optical instruments such as Scanning Electron Microscope, Confocal Microscope, Fluorescence Microscope, or Optical Microscope have been used.

In this regard, as a part of efforts to identify the physical characteristics of cytoplasm, there have been some examples such as measuring the viscoelasticity of cytoplasm by using magnetic materials (Crick, et. al. The physical properties of cytoplasm: a study by means of the magnetic particle method. Exp. Cell Res. (1950) 1, 505-533) and measuring the rheology of actin filament fluid of cytoplasm (Ziemann, et. al. Local measurements of the viscoelastic moduli of entangled actin networks using an oscillating magnetic bead micro-rheometer. Biophys. J. (1994) 66, 2210-2216). However, the efforts to trace the movement of the cell by using magnetic materials, to identify the cellular structure and metabolisms by using the movement of magnetic materials, or to identify biological characteristics of cytoplasm have been sluggish.

Meanwhile, as a part of another efforts to understand cellular structure and metabolisms, a fluorescent probe technology for labeling a particular material with fluorescence followed by observing it under a microscope has been developed and been used (Lippincott-Schwartz, et al. Studying protein dynamics in living cells. Nat. Rev. Mol. Cell. Biol. (2001) 2, 444-456; Zhang, et al. Creating new fluorescent probes for cell biology. Nat. Rev. Mol. Cell. Biol. (2002) 3, 906-918).

The fluorescent probe technology has an advantage to recognize the location of a particular material inside a cell without cell lysis. However, it has some disadvantages to be limited to identify various biological phenomena inside a cell and metabolisms and to trace materials involved in signal transduction. In addition, a fluorescent probe technology using different types of nanoparticles or nanocrystals, a cell-labeling technology, and a tracking technology for subcellular organelles have been recently developed (Chan, et al. Luminescent quantum dots for multiplexed biological detection and imaging. Curr. Opin. Biotechnol. (2002) 13, 40-46; Berry & Curtis. Functionalization of magnetic nanoparticles for applications in biomedicine. J. Phys. D Appl. Phys. (2003) 36, R198-R206; Rudin & Weissleder. Molecular imaging in drug discovery and development. Nat. Rev. Drug Discov. (2003) 2, 123-131; Alivisatos. The use of nanocrystals in biological detection. Nat. Biotechnol. (2004) 22, 47-52; Derfus, et al. Intracellular delivery of quantum dots for live cell labeling and organelle tracking Adv. Mater. (2004) 16, 961-966). But, they still have the disadvantages as stated above.

As an alternative way to solve those difficulties, magnetic materials have been noted and tried for the research and medical use (Alexiou, et al. Locoregional cancer treatment with magnetic drug targeting. Cancer Res. (2000) 60, 6641-6648; Lewin, et al. Tat peptide-derived magnetic nanoparticles allow in vivo tracking and recovery of progenitor cells. Nat. Biotechnol. (2000) 18, 410-414; Berry & Curtis. Functionalization of magnetic nanoparticles for applications in biomedicine. J. Phys. D Appl. Phys. (2003) 36, R198-R206; Beckmann, et al. Magnetic resonance imaging in drug discovery: lessons from disease areas. Drug Discov. Today (2004) 9, 35-42; Kelloff, et al. The progress and promise of molecular imaging probes in oncologic drug development. Clin. Cancer Res. (2005) 11, 7967-7985).

For example, the use of magnetic materials has been tried to split cells and separate a particular material from cell lysate (Saiyed, et al. Application of magnetic techniques in the field of drug discovery and biomedicine. BioMagnetic Res. Technol. (2003) 1, 2). However, researches for introducing magnetic materials into a cell and manipulating them effectively followed by observing their intracellular movement, and thus identifying cellular structure and metabolisms are still immature because of the limited mobility of the magnetic materials and the related characteristics of cytoplasm inside a cell.

In this regard, trials to measure physical properties of cell surface receptors by using the intracellular movement of magnetic materials on the cell surface, to measure the viscosity of cytoplasm by using the movement of magnetic materials inside a cell by applying a magnetic field, and to move magnetic materials to a specific site in a cell have been reported (U.S. Pat. No. 5,486,457; Gehr, et. al. Magnetic particles in the liver: a probe for intracellular movement. Nature (1983) 302, 336-338; Valberg, Pa. Magnetometry of ingested particles in pulmonary macrophages. Science (1984) 224, 513-516; Valberg & Feldman. Magnetic particle motions within living cells: measurement of cytoplasmic viscosity and motile activity. Biophys. J. (1987) 52, 551-561; Andreas, et. al. Measurement of Local Viscoelasticity and Forces in Living Cells by Magnetic Tweezers, Biophys. J. (1999) 76, 573-579; Gao, et al. Intracellular spatial control of fluorescent magnetic nanoparticles. J. Am. Chem. Soc. (2008) 130, 3710-3711). But they have been regarded as insufficient in terms of introducing magnetic materials effectively into a cell followed by manipulating and monitoring their movement in a cell.

More specifically, as the diameter of early endosome and late endosome in a general cell such as a HeLa cell except macrophage-like cells is on average 200˜300 nm and 750 nm, respectively, it is difficult to introduce magnetic materials having very large diameter into a cell (Lodish, et. al. op. cit. 727; Brandhorst, et. al. Homotypic fusion of early endosomes: SNAREs do not determine fusion specificity. Proc. Natl. Acad. Sci. USA (2006) 103, 2701-2706; Ganley, et. al. Rab9 GTPase regulates late endosome size and requires effector interaction for its stability. Mol. Biol. Cell (2004) 15, 5420-5430).

On the other hand, since the resolution of an optical microscope is approximately 200 nm, when magnetic materials having small diameter of several nanometers or several tens of nanometers are separated independently, it is difficult to recognize their location inside a cell. For example, among subcellular organelles, vesicles having a sphere-like shape such as endosome and lysosome may be observed as black dots scattered sporadically under an optical microscope and hence it is extremely difficult to distinguish small magnetic particles observed as black dots from subcellular organelles and to monitor them in a cell.

The inventor of the present invention has developed a method for introducing magnetic materials into a live cell and patterning magnetic materials in a direction of a line of magnetic force in a live cell by applying a magnetic field, a method for imaging a pattern of magnetic materials in a direction of a line of magnetic force by a label and an apparatus used for the same.

### SUMMARY OF INVENTION

The object of the present invention is to provide a method for patterning magnetic materials introduced into a live cell, in a magnetized direction induced by applying a magnetic field, a method for imaging a pattern of magnetic materials and an apparatus used for the same.

In addition, the object of the present invention is to provide a method for patterning magnetic materials introduced into a live cell, in a direction of a line of magnetic force by applying a magnetic field, a method for imaging a pattern of magnetic materials in a direction of a line of magnetic force by a label and thus a technology capable of easily monitoring cellular structures and metabolisms in a live cell.

### DETAILED DESCRIPTION OF INVENTION

The present invention provides a method for patterning magnetic materials in a live cell comprising: preparing a plurality of magnetic materials magnetized in a direction of a line of magnetic force by applying a magnetic field, at least one of the magnetic materials being configured into a nanoparticle and the surface of at least one of the magnetic materials being modified; introducing a plurality of the magnetic materials into each live cell; allowing a bundle of the lines of magnetic force to pass through the live cell in a direction by applying a focused magnetic field to the live cell; aligning a plurality of the magnetic materials in the live cell with the direction of the line of magnetic force by the applied magnetic field; and identifying a pattern in which a plurality of the magnetic materials are aligned with the direction of the line of magnetic force.

In addition, the present invention provides a method for imaging a pattern of magnetic materials in a live cell comprising: preparing a plurality of magnetic materials magnetized in a direction of a line of magnetic force by applying a magnetic field, at least one of the magnetic materials being configured into a nanoparticle and the surface of at least one of the magnetic materials being modified; introducing a plurality of the magnetic materials into each live cell; providing the live cell with a label capable of binding to the magnetic material and imaging a pattern of the magnetic materials in a direction of a line of magnetic force; allowing a bundle of the lines of magnetic force to pass through the live cell in a direction by applying a focused magnetic field to the live cell; aligning a plurality of the magnetic materials in the live cell with the direction of the line of magnetic force by the applied magnetic field; and identifying an imaged pattern of the label capable of imaging the aligned pattern of the magnetic materials.

In one embodiment of the present invention, the magnetic materials should be materials capable of being magnetized by applying a magnetic field. For example, the magnetic material may be a transition metal compound selected from a group consisting of period 4 transition metals such as iron, manganese, chrome, nickel, cobalt, and zinc; their oxides, sulfides, and phosphides; their alloys; and oxides, sulfides, and phosphides of the alloys, or a composition including at least one of them.

Preferably, the magnetic material may include one or mixture of at least two selected from a group consisting of magnetite (Fe₃O₄), maghemite (gamma-Fe₃O₄), cobalt ferrite (CoFe₂O₄), manganese oxide (MnO), manganese ferrite (MnFe₂O₄), iron (Fe)-platinum (Pt) alloy, cobalt (Co)-platinum (Pt) alloy and cobalt (Co).

In one embodiment of the present invention, the diameter of the magnetic material is about 1˜1,500 nm, preferably, about 20˜350 nm.

In one embodiment of the present invention, the saturation magnetization of the magnetic material is preferably above 40 emu (electromagnetic unit)/g, and the magnetic material may have the feature of superparamagnetism or ferromagnetism.

In one embodiment of the present invention, the magnetic material introduced into the live cell can be observed as a black dot under an optical microscope and the diameter of the black dot is preferably above 150˜3,000 nm. Considering the theoretical resolution of an optical microscope is approximately 200 nm (Lodish, et. al. Molecular Cell Biology 4th ed. W. H. Freedman and company, (2000) 140-141), it is desirable that the diameter of the black dot is above 300˜1,500 nm. The black dot may comprise a single magnetic material or a plurality of magnetic materials locally adjacent to each other.

Meanwhile, if the magnetic material includes a fluorescent material such as RITC (Rhodamine B isothiocyanate) or FITC (fluoresceine isothiocyanate), the magnetic material in the live cell can be observed as a fluorescence dot emitting its own light under a fluorescence microscope and in this case, the diameter of the fluorescence dot may be observed to be larger than that of the magnetic material.

In one embodiment of the present invention, a plurality of black dots may exist in the live cell.

In one embodiment of the present invention, when the focused magnetic field is applied to the live cell, the magnetic field can be applied in a horizontal direction to the bottom on which the live cell is placed.

In one preferable embodiment of the present invention, the step of applying the focused magnetic field to the live cell is performed by an apparatus for applying a magnetic field. The apparatus for applying a magnetic field comprises a cylindrical core consisting of unmagnetized magnetic materials for strengthening the magnetic field and for fixing a container in which the live cell is placed, or a means for increasing the magnetic field gradient provided with a plurality of extensions that support the container, which makes it easy to magnetize the magnetic materials in the direction of the line of magnetic force by focusing the magnetic field to the container and thus strengthening a force to move and maintain the magnetic materials in a certain direction to the container. In addition, a permanent magnet or an electromagnet may be preferably located adjacent to the cell.

One embodiment of the present invention, the method further comprises identifying the pattern of the magnetic materials and the pattern imaged by the label, and whether the pattern imaged by the label is co-localized with the pattern of the magnetic materials or not.

In one embodiment of the present invention, the magnetic material can be labeled with the label through a mediator. The mediator may include a single linker or a plurality of linkers. For example, the mediator may consist of two linkers.

In one embodiment of the present invention, the magnetic material can be labeled with the label through the mediator before introducing the magnetic material and the label into the live cell.

Alternatively, the magnetic material can be labeled with the label through the mediator after introducing the magnetic material and the label into the live cell, separately. If it is identified that the pattern of the magnetic materials is co-localized with the pattern imaged by the label, it can be considered that the linkers of the mediator bind to each other in the live cell.

In one embodiment of the present invention, the linkers of the mediator may include macromolecules such as proteins, nucleic acids, polysaccharides and lipids, and small molecules such as amino acids, nucleotides, phosphoric acids, vitamins, amines and other organic compounds.

Meanwhile, an apparatus used for the aforesaid method comprises: a container for culturing a live cell provided with a plurality of magnetic materials magnetized in a direction of a line of magnetic force by applying a magnetic field, and a label capable of binding to the magnetic material and imaging a pattern of the magnetic materials in a direction of a line of magnetic force, at least one of the magnetic materials being configured into a nanoparticle and the surface of at least one of the magnetic materials being modified; an apparatus for allowing a bundle of the lines of magnetic force to pass through the live cell in a direction by applying a focused magnetic field to the live cell; and a device for monitoring the pattern of the magnetic materials aligned with the direction of the line of magnetic force and/or the imaged pattern of the label capable of imaging the aligned pattern of the magnetic materials in the live cell.

In one embodiment of the present invention, the device can identify whether the pattern of the magnetic materials is co-localized with the pattern imaged by the label or not.

In one preferable embodiment of the present invention, the apparatus comprises a cylindrical core consisting of unmagnetized magnetic materials for strengthening the magnetic field and for fixing the container in which the live cell is placed, or a means for increasing the magnetic field gradient provided with a plurality of extensions that support the container, which makes it easy to magnetize the magnetic materials in the direction of the line of magnetic force by focusing the magnetic field to the container and thus strengthening a force to move and maintain the magnetic materials in a certain direction to the container.

### ADVANTAGEOUS EFFECTS

According to the present invention, it is possible to pattern magnetic materials introduced into a live cell, in a direction of a line of magnetic force by applying a magnetic field, and to image a pattern of magnetic materials in a direction of a line of magnetic force by a label.

In addition, according to the present invention, cellular structures and metabolisms can be easily monitored in a live cell by imaging a pattern of magnetic materials introduced into a live cell in a direction of a line of magnetic force by a label.

### BRIEF DESCRIPTION OF DRAWINGS

The above and other objects, features and other advantages of the present invention will be more clearly understood to those skilled in this arts from the following detailed description taken in conjunction with the accompanying drawings.

FIG. 1 represents a picture taken from a scanning electron microscope showing magnetic materials synthesized by a method of one embodiment of the present invention.

FIG. 2 represents a picture taken from a transmission electron microscopy showing magnetic materials synthesized by a method of one embodiment of the present invention.

FIG. 3 represents a perspective view of one apparatus for applying a magnetic field used for the present method in one embodiment of the present invention.

FIG. 4 represents a perspective view of another apparatus for applying a magnetic field (equipping with a means of increasing a magnetic field gradient) used for the present method in one embodiment of the present invention.

FIG. 5(A) is a photograph taken from a transmitted light microscopy after fixing HeLa cell into which magnetic materials were introduced without applying a magnetic field. FIG. 5(B) is a photograph taken from a transmitted light microscopy after applying a magnetic field in a vertical direction and fixing HeLa cell into which magnetic materials were introduced. FIG. 5(C) is a photograph taken from a transmitted light microscopy after applying a magnetic field in a horizontal direction and fixing HeLa cell into which magnetic materials were introduced.

FIG. 6 illustrates and compares pictures taken from an optical microscope after applying a magnetic field to HeLa cells into which magnetic materials were introduced and fixing them (magnetic field +) and after fixing them without applying a magnetic field (magnetic field −). The left image is a transmitted light image with original color before Prussian blue staining and the right image is a transmitted light image after fixing and Prussian blue staining of HeLa cells. The arrow indicates a direction of a line of magnetic force in a horizontal direction.

FIG. 7 represents pictures taken from a fluorescence microscopy and a transmitted light microscopy, showing that magnetic materials introduced into cells form a pattern in accordance with a direction of a magnetic field. The arrow indicates a direction of a line of magnetic force.

FIG. 8 illustrates and compares pictures taken from a fluorescence microscopy and a transmitted light microscopy, after applying a magnetic field to HeLa cells into which the fluorescence-labeled magnetic particles complex were introduced and fixing them (magnetic field +) and after fixing them without applying a magnetic field (magnetic field −).

FIG. 9 represents a schematic diagram showing processes for synthesizing dasatinib-biotin.

FIG. 10(A) illustrates and compares pictures taken from a fluorescence microscopy and a transmitted light microscopy, after applying a magnetic field to HeLa cells into which Dasatinib-MNP complex (Das-MNP) or Biotin-MNP complex (Bio-MNP) and CSK-EGFP expression vector were introduced.

FIG. 10(B) illustrates and compares pictures taken from a fluorescence microscopy and a transmitted light microscopy, after applying a magnetic field to HeLa cells into which Dasatinib-MNP complex (Das-MNP) or Biotin-MNP complex (Bio-MNP) and SNF1LK-EGFP expression vector were introduced.

### EXAMPLES

Practical and presently preferred embodiments of the present invention are illustrated more clearly as shown in the following examples. However, it should be appreciated that those skilled in the art, on consideration of this disclosure, may make modifications and improvements within the spirit and scope of the present invention. References cited in the specification are incorporated into the present invention.

### Example 1

### The Synthesis of Magnetic Material

(1) The Synthesis of Magnetic Particle In one embodiment of the magnetic materials capable of forming a pattern in a cell by applying a magnetic field, magnetic particles were synthesized and used by a modified method from that of Molday et. al. as the following (Molday, et. al. Immunospecific ferromagnetic iron-dextran reagents for the labeling and magnetic separation of cells. J. Immunol. Meth. (1982) 52, 353-367).

50 ml conical tube (Cat. No. 50050 from SPL Lifesciences at Pocheon, Gyeonggi-do, South Korea) is located in a ultrasonic cleaning bath capable of regulating temperature and ultrasonic intensity (Model No. JAC2010 from Kodo Technical Research Co., Ltd.) and 10 ml of 50% dextran solution (w/w) (Fluka, Cat. No. 31389; average molecular weight of 40,000) is added to the tube.

A propeller connected to a laboratory stirrer (Model No. PL-S10 from Poonglim trading company at Jangsa-dong, Jongro-gu, Seoul, South Korea) is equipped in the conical tube and stirred at approximately 2,000 r.p.m. while a solution of 10 ml of 1.51 g FeCl₃.6H₂O (Sigma, Cat. No. 236489) and 0.64 g FeCl₂.4H₂O (Sigma, Cat. No. 220299) is added dropwise to the tube. In this process, ultrasonic waves may be applied to the solution.

While stirred, 7.5% NH₄OH (v/v) is added dropwise to the solution in order to adjust pH to 10.5. In this step, the temperature of a water bath may be adjusted to 40-65° C.

When the pH of the solution reaches to 10.5, the solution is boiled for 15 minutes in a water bath adjusted to 65-90° C. beforehand. The boiled solution is left to decrease its temperature gradually at room temperature.

After centrifugation at 600×g for 5 mins three times using a centrifuge (Cat. No. MF-80 from Hanil Science Industrial Inc. at Jakjeon-dong, Gyeyang-gu, Incheon, South Korea), the supernatant is collected and then centrifuged at 2,000×g for 10 mins to remove the precipitate.

After centrifugation at 3,900 r.p.m. for 20 mins using a tabletop centrifuge (Hanil Science Industrial Inc. Model No. Combi 514K) to remove unbound form of dextran through Amicon Ultracel-100K (Millipore, Cat. No. UFC910024), magnetic particles are floated in purified water, phosphate buffered saline, or 2 M sodium carbonate (Na₂CO₃) solution (pH 11). The unbound form of dextran may be also removed by a gel filtration chromatography using Sephacryl S-300.

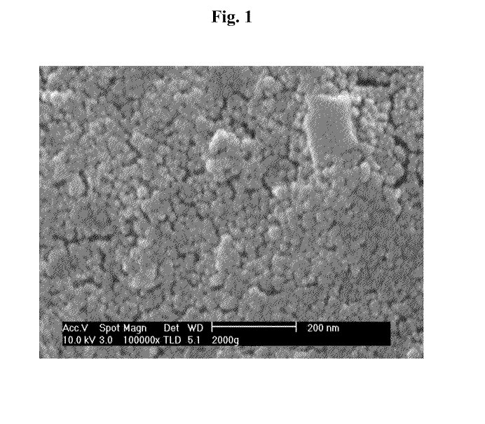

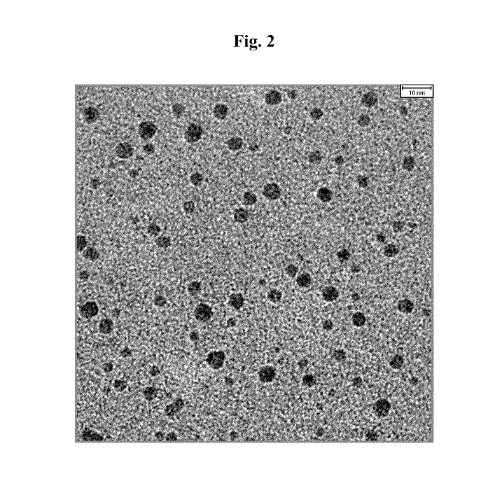

FIG. 1 is a picture taken from a scanning electron microscope showing magnetic materials synthesized by the aforesaid method and FIG. 2 is a picture taken from a transmission electron microscopy showing magnetic materials synthesized with no addition of dextran by a modified method from the aforesaid method.

(2) The Surface Modification of Magnetic Particle

To modify the surface of magnetic particle synthesized by the above method (1) with streptavidin or proteins such as fluorescent proteins, a well-known method in the art was modified and used (March, et. al. A simplified method for cyanogen bromide activation of agarose for affinity chromatography. Anal. Biochem. (1974) 60, 149-152; Cuatrecasas, P. Protein purification by affinity chromatography. J. Biol. Chem. (1970) 245, 3059-3065). The surface modification of magnetic particle is as follows.

To activate hydroxyl group exposed to the surface of magnetic particle, 5 M CNBr solution (a solution of 2 g cyanogen bromide dissolved in 1 ml of acetonitril) was added to magnetic particles floated in 2 M sodium carbonate (Na₂CO₃) solution (pH 11) as prepared in the above method (1) to 2% of a total volume in a chemical hood.

To make a reaction slow, the reaction was performed at relatively low temperature of 4-20° C. for 8-12 mins. Unreacted CNBr (cyanogen bromide) was removed by a well-known method in the art such as dialysis, centrifugation, HGMS (High gradient magnetic separation) technology (U.S. Pat. No. 4,247,398; Melville, et. al. Direct magnetic separation of red cells from whole blood. Nature (1975) 255, 706), and ultrafiltration.

After activated magnetic particles with no unreacted CNBr were floated in phosphate buffered saline, or 0.1 M sodium bicarbonate solution, they were then mixed with 10˜200 mg/ml of protein solution diluted in phosphate buffered saline and reacted at 4° C. for 14 hours to allow the surface of magnetic particle to be modified by the protein. The final concentration of the protein was adjusted to 0.1˜100 mg/ml. Preferably, the final concentration of the reactive protein was adjusted to 1˜10 mg/ml.

To terminate the surface modification of magnetic particle by the protein, glycine was added to 5˜50 times of the final protein concentration and reacted at room temperature for 2 hours. Preferably, glycine was added to 10˜25 times of the final protein concentration.

Unreacted proteins and glycines were removed by a well-known method in the art such as centrifugation and HGMS.

By the method mentioned above, a streptavidin-magnetic particle was prepared by modifying the surface of magnetic particle with streptavidin, and EGFP (enhanced green fluorescence protein)-magnetic particle was prepared by modifying the surface of magnetic particle with EGFP which is one of fluorescent proteins widely known in the art.

It could be confirmed by SDS-discontinuous polyacrylamide gel electrophoresis (SDS-PAGE) whether the surface of magnetic particle was modified by the protein or not.

A surface-modified magnetic particle by a protein available in the present invention can be produced directly by a well-known method in the art (U.S. Pat. No. 5,665,582; U.S. Patent Publication No. 2003/0092029A1) or purchased from a company responsible for production and sales.

### Example 2

Patterning Magnetic Materials Introduced into a Cell in a Direction of a Line of Magnetic Force

HeLa cells (purchased from ATCC, Cat. No. CCL-2) were subcultured to 5,000 cells/well in a 96-well plate, and cultured in DMEM media containing 10% fetal bovine serum (Invitrogen) in an incubator at a 37° C., 5% CO₂.

On the next day, after a peptide having a protein transduction domain (PKKKRKVGLFGAIAGFIENGWEGMIDG) was added to magnetic materials synthesized in Example 1 and adjusted to 0.1 mM of the final concentration, the reaction was performed at room temperature for 30 mins and the unbound form of the protein transduction domain was removed by HGMS method.

In one embodiment of the present invention, it is easily understood by an ordinarily skilled person in the art that other protein transduction domains known in the art and a peptide called “penetratin” can be used as a protein transduction domain as well as the sequence.

Next, after cultured HeLa cells were washed with 1×D-PBS, OPTI MEM I medium (Invitrogen) containing the magnetic materials and the protein transduction domain was added to the cells, and then the cells were cultured in an incubator at a 37° C., 5% CO₂.

On the next day, the magnetic material-treated cells were washed twice with OPTI-MEM I medium (Invitrogen, Cat. No. 31985-070) and a magnetic field was applied to the cells in OPTI-MEM I medium using a permanent magnet or an electromagnet according to a well-known method (KR Patent No. 10-0792594; KR Patent No. 10-0862368; and U.S. Pat. No. 4,247,398).

Due to the viscosity of cytoplasm and the cellular structure in the cells, the magnetic materials inside the cells should be treated with stronger magnetic force than in a solution to move them in the cells by applying a magnetic field.

A magnetic force influencing magnetic materials in a cell is directly proportional to magnetization and magnetic flux density. As the magnetic flux density of magnetic field is influenced by the saturated magnetization of metals constituting a magnet, a magnet should be made of metals with high saturated magnetization to produce high magnetic flux density.

As the magnetic flux density is inversely proportional to the square of a distance from a magnet to a cell, a magnet should be as close as possible to a cell in order to provide magnetic materials in a cell with high magnetic flux density.

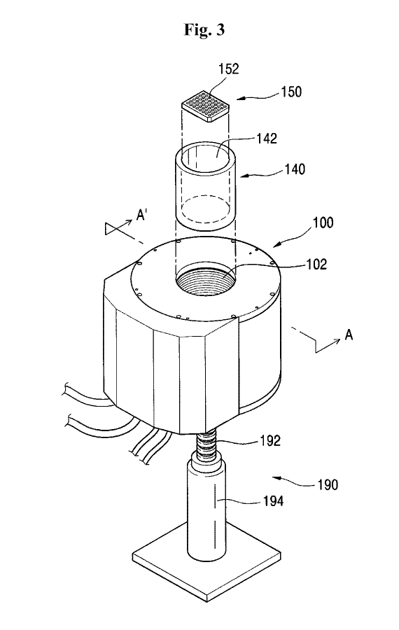

In this regard, FIG. 3 illustrates an apparatus for applying a magnetic field used in one embodiment of the present invention (KR Patent No. 10-0792594). The apparatus is equipped with a body 100 for receiving a well plate 150 with a number of wells 152, and a core 140 consisting of unmagnetized magnetic materials for strengthening a magnetic field and for fixing the well plate 150. Though not illustrated herein, the apparatus also has coils wound on the body 100 from several times to hundreds of thousands of times to form a magnetic field in areas which include the well plate 150 when supplying electric power, and a power supply to provide the coils with the electric power.

The core 140 consists of unmagnetized material materials which are magnetized only when an electric current flows through the core. The magnetic field is focused on the core 140 by providing the apparatus with the core 140 to increase the intensity of the magnetic field near the core 140. Therefore, as the magnetic field is focused near the core 140 by providing the apparatus with the core 140 to rapidly increase the intensity of the magnetic field, a force for moving and maintaining the magnetic materials in the well-plate 150 is strengthened and the magnetic materials can be easily patterned in a direction of a line of magnetic force.

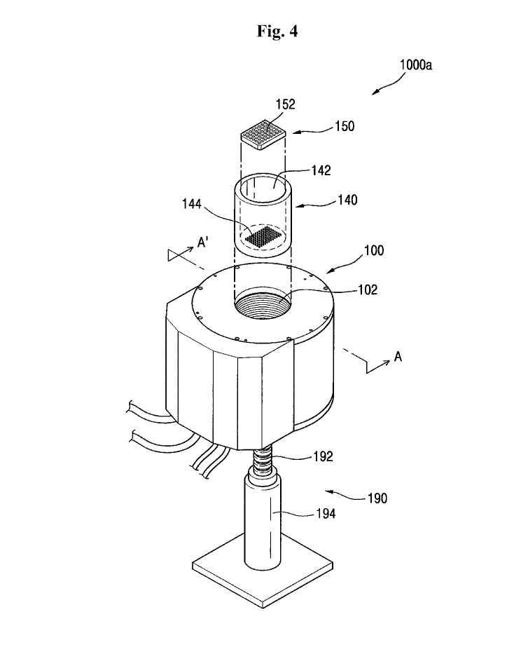

In addition, FIG. 4 illustrates another apparatus for applying a magnetic field used in one embodiment of the present invention (KR Patent No. 10-0862368). The apparatus 1000a comprises a body 100 for receiving a well plate 150 with a number of wells 152, and a means for increasing a magnetic field gradient 144 provided with a plurality of extensions that support the well plate 150 and protrudes upward from the base of the body 100. Though not illustrated herein, the apparatus also has coils wound on the body 100 from several times to hundreds of thousands of times to form a magnetic field in areas which include the well plate 150 when supplying electric power, and a power supply to provide the coils with the electric power.

As the means for increasing a magnetic field gradient 144 increases a gradient of magnetic field by a plurality of extensions, a force for moving and maintaining the magnetic materials inside each well 152 of the well-plate 150 is strengthened and the magnetic materials can be easily patterned in a direction of a line of magnetic force.

Under a magnetic field, magnetic materials were washed once using 1×D-PBS (Welgene, Cat. No. LB001-02) and 37% formaldehyde (Sigma) was 10-fold diluted in 1×D-PBS to prepare 3.7% formaldehyde solution. Cells were fixed after treating the cells with the formaldehyde solution for 5 mins, followed by being washed with 1×D-PBS three times, and then were observed by a microscope.

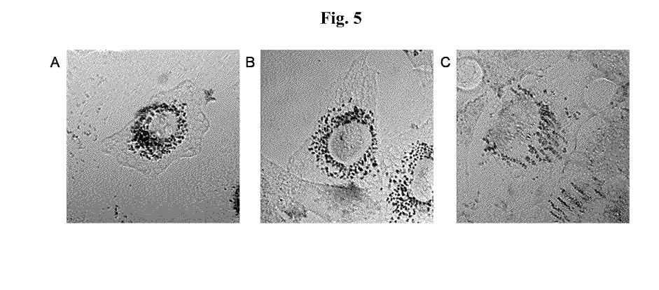

In this embodiment, Olympus fluorescence microscope, FV1000, equipped with the object lens of Uplan Apo 40×/0.85 was used to obtain transmitted images in the cells. The result is illustrated in FIG. 5. As shown in FIG. 5(A), in cells without applying a magnetic field, the magnetic materials around their nuclei were identified but a pattern in a direction of a line of magnetic force was invisible. In addition, in cells applied by a magnetic field in a vertical direction, the magnetic materials around their nuclei were also identified, but a pattern in a direction of a line of magnetic force was invisible (see FIG. 5(B)). Meanwhile, in cells applied by a magnetic field in a horizontal direction, a pattern in a direction of a line of magnetic force was apparently observed (see FIG. 5(C)). This result means that magnetic materials form a pattern inside a cell in a direction of a line of magnetic force by induced magnetization.

Among subcellular organelles, vesicles having a sphere-like shape such as endosome and lysosome may be observed as black dots scattered sporadically under an optical microscope. Therefore, to identify whether magnetic materials form a pattern in a direction of a line of magnetic force, a plurality of magnetic materials should be introduced into each cell effectively and a bundle of lines of magnetic force should be able to pass through the cell in a direction by applying a focused magnetic field. In this embodiment of the present invention, the pattern was identified by black dots aligned with a direction of a line of magnetic force, clearly distinguished from subcellular organelles having sphere-like shape.

### Example 3

### Identifying a Pattern of Magnetic Materials Introduced into a Cell in a Direction of a Line of Magnetic Force Using Prussian Blue Dye

As shown in FIG. 5, Prussian blue staining was performed to identify if black dots observed from a transmitted light microscopy were magnetic materials. In general, Prussian blue dye is used to specifically stain oxidized iron particles introduced into a cell and to observe the oxidized iron particles (Frank, J. A., Miller, B. R., Arbab, A. S., Zywicke, H. A., Jordan, E. K., Lewis, B. K., Bryant, L. H., & Bulte, J. W. M. (2003) Clinically applicable labeling of mammalian and stem cells by combining superparamagnetic iron oxides and transfection agents. Radiology 228: 480-487). Cells into which magnetic materials were introduced, were prepared as mentioned in Example 2. After applying a magnetic field for 3 hours, cells were fixed with formaldehyde. The cell was observed by an optical microscope before and after Prussian blue staining in the same field. Prussian blue staining was performed by Prussian Blue Iron Stain Kit (Polysciences, Cat. No. 24199).

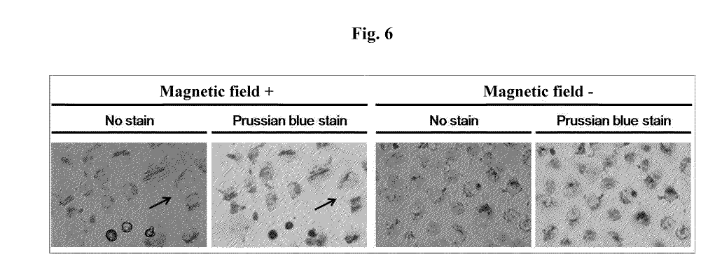

In this embodiment, Olympus IX51 inverted system microscope equipped with the object lens of LUCPlan F1 60× (Olympus IX51 inverted system microscope equipped with DP70 CCD camera, Japan) was used to obtain transmitted images in cells. As shown in FIG. 6, it was observed under an optical microscopy that a pattern of magnetic materials in cells was formed in a direction of a line of magnetic force (in an arrow direction) by applying a magnetic field to cells. Further, it was observed that the pattern was specifically stained by Prussian blue staining. Therefore, it was confirmed that the pattern formed by applying a magnetic field was induced by the magnetic materials introduced into the cells. In contrast, it was observed that the magnetic materials introduced into the cells was stained by Prussian blue staining without applying a magnetic field. However, a pattern was not formed in the cells in a direction of a line of magnetic force without applying a magnetic field.

### Example 4

### Observing the Change of the Pattern of the Magnetic Materials by Changing Magnetic Orientation

The change of the pattern of the magnetic materials was identified by changing magnetic orientation as follows.

Cells into which magnetic materials were introduced, were prepared as mentioned in Example 2. As described in Example 2, a magnetic field was applied to live cells and then the cells were fixed. In this embodiment, Zeiss LSM510 META NLO microscope equipped with the object lens of Plan-Neofluar 20× was used to obtain fluorescence and transmitted images in the cells.

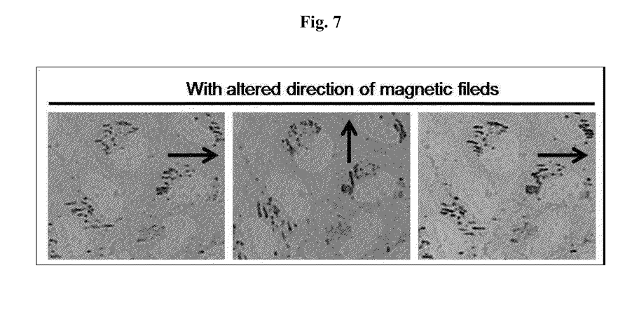

As shown in FIG. 7, a pattern of magnetic materials was observed in a direction of a line of magnetic force (in an arrow direction) by applying a magnetic field, and this pattern was identified to be remodeled in accordance with the magnetic direction.

### Example 5

Observing the Fluorescent Pattern of Fluorescence-Labeled Magnetic Materials in a Direction of a Line of Magnetic Force

To observe a magnetic pattern of fluorescence-labeled magnetic materials being imaged by a fluorescent label, the following experiment was conducted.

HeLa cells (ATCC, Cat. No. CCL-2) were subcultured to 4,000 cells/well in a 96-well plate (Greiner, Cat. No. 655090).

On the next day, magnetic materials were treated to the cultured HeLa cells as follows:

1) Streptavidin-magnetic particles were mixed with biotin-SS-FITC, being allowed to be reacted, and then the magnetic particles were labeled with fluorescence;

2) After 30 mins, the mixture was purified by a well-known separating method of magnetic materials in the art (e.g., HGMS method); and

3) As mentioned in Example 2, the purified magnetic particles labeled with fluorescence were treated to the cells, and then after applying a magnetic field the cells were fixed with a formaldehyde solution.

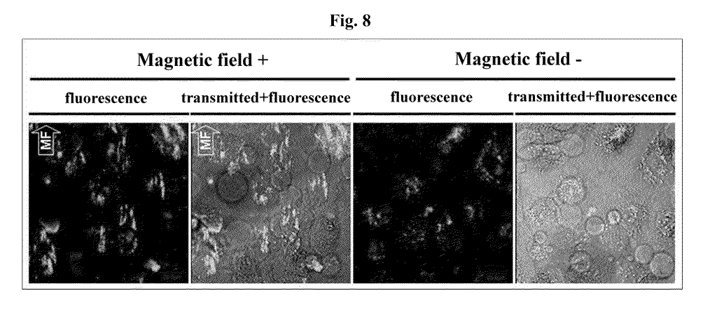

In this embodiment, Olympus fluorescence microscope, FV1000, equipped with the object lens of Uplan Apo 40×/0.85 was used to obtain fluorescent and transmitted images in the cells. As shown in FIG. 8, the fluorescent pattern of magnetic materials fluorescently labeled with FITC was identified in a direction of a line of magnetic force (in an arrow direction) by applying a magnetic field. On the other hand, in cells without applying a magnetic field, the FITC fluorescence was observed, but a specific fluorescent pattern was invisible. In this embodiment, therefore, it was identified that the magnetic pattern of a plurality of the magnetic particles formed in the cell by applying a focused magnetic field was imaged by a fluorescent material as a label.

### Example 6

### Observing the Fluorescent Pattern of Fluorescence-Labeled Magnetic Materials in a Direction of a Line of Magnetic Force Using a Mediator

In this embodiment, it was observed whether a magnetized pattern of a plurality of magnetic materials formed by applying a magnetic field could be imaged by fluorescent labels when a surface-modified magnetic material was labeled with a fluorescent label through a mediator indirectly as well as directly. In addition, it was identified whether a fluorescent pattern was formed and overlapped (co-localized) with a pattern formed by magnetic materials in a direction of a line of magnetic force or not.

A mediator may include a single linker or a plurality of linkers. In the following example, an experiment was performed using a mediator consisting of two linkers. The surface-modified magnetic particle can be labeled with a fluorescent material through a mediator before or after introducing the magnetic particle and the fluorescent material into a cell.

Dasatinib which has been used to treat chronic myeloid leukemia was selected for one linker constituting a mediator [Lombardo, L. J., Lee, F. Y., Chen, P., et al. Discovery of N-(2-chloro-6-methyl-phenyl)-2-(6-(4-(2-hydroxyethyl)-piperazin-1-yl)-2-methylpyrimidin-4-ylamino)thiazole-5-carboxamide (BMS-354825), a dual Src/Abl kinase inhibitor with potent antitumor activity in preclinical assays. J. Med. Chem. 47, 6658-6661 (2004); Shah, N. P., Tran, C., Lee, F. Y. Chen, P., Norris, D., Sawyers, C. L. Oerriding imatinib resistance with a novel ABL kinase inhibitor. Science 305, 399-401 (2004)].

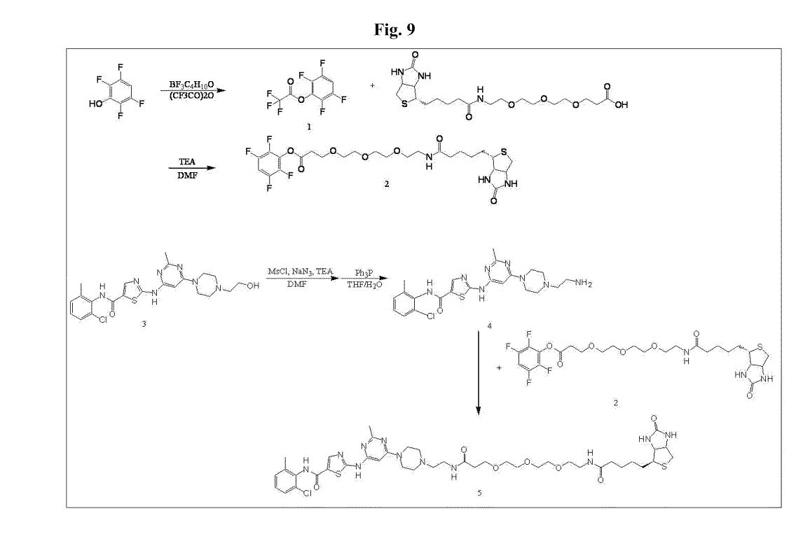

In addition, CSK (GenBank Acc. No. NM_{—}004383.1) or SNF1LK (GenBank Acc. No. BCO38504) which binds to dasatinib was used for another linker constituting a mediator. Next, to modify the surface of magnetic particle with dasatinib, dasatinib-biotin was synthesized by a known method in the art, for example, as shown in FIG. 9.

For example, the process of dasatinib-biotin synthesis is explained as follows. To synthesize dasatinib-biotin, biotin linker was firstly synthesized in a form of 2,3,5,6-tetrafluorophenyl trifluoroacetate: (1) Under atmosphere of nitrogen, BF₃ ethylether was added to 2,3,5,6-tetrafluorophenol dissolved in trifluoroacetic anhydride; and (2) After removing the solvent, the biotin linker compound was coupled with compound 1 dissolved in TEA/DMF mixture to produce compound 2.

Dasatinib-biotin was then synthesized as follows: (1) After dasatinib was dissolved in a mixture of THF and DMF and then triethylamine was added, the dasatinib solution was cooled; (2) Methanesulfonyl chloride was slowly and dropwise added to the dasatinib solution and stirred overnight at room temperature; (3) After adding NaN₃, the reaction solution was stirred overnight at 50° C.; (4) The reaction solution was subjected to vacuum evaporation and then its residue was purified by column chromatography; (5) The purified product was dissolved in THF and after adding an excess of triphenylphosphine, it was stirred for 5 hours at room temperature; (6) After adding water to the reaction solution, the solution was stirred overnight at 70° C. and then subjected to vacuum evaporation; (7) The residue was dissolved in DMF under atmosphere of nitrogen, and then triethylamine was added to the solution; (8) Compound 2 dissolved in DMF was added to the solution, and the solution was stirred for 3 days at room temperature; and (9) The reaction solution was subjected to vacuum evaporation and then purified by MeOH/MC column chromatography.

The synthesized compound was identified by NMR or LC-MS.

CSK cDNA (GenBank Acc. No. NM_{—}004383.1) was purchased from Open Biosystems. To make a CSK OFR clone from which a stop codon is removed, the following experiment was conducted. Primers for amplifying a CSK ORF were purchased from Cosmo Genetech Inc. (South Korea) and their sequences are the followings: CSK-F primer, 5′-GCA GGC TCC ACC ATG TCA GCA ATA CAG GCC GCC T-3′; CSK-R primer, 5′-CAA GAA AGC TGG GTG CAG GTG CAG CTC GTG GGT TTT G-3′. CSK cDNA was used as a template and the primers were used for the following PCR amplification: 95° C., 5 mins, 1 cycle; 95° C., 0.5 min, 50° C., 0.5 min, 72° C., 2 mins, 1030 cycles; 72° C., 7 mins, 1 cycle. The DNA polymerase used for the PCR amplification was purchased from Stratagene, Enzynomics, Cosmo Genetech, or ELPIS Biotech, etc. and used according to a manual of the manufacturer. The amplified CSK ORF was re-amplified using the following primers: attB1-F2 primer, 5′-GGGGACAAGT TTGTACAAAA AAGCAGGCTC CACCATG-3′; attB2-R2 primer, 5′-GGGGACCACT TTGTACAAGA AAGCTGGGTG-3′. The PCR product of the CSK ORF was used as a template and attB1-F2 and attB2-R2 primers were used for the following PCR amplification: 95° C., 2 mins, 1 cycle; 95° C., 0.5 min, 45° C., 0.5 min, 72° C., 2 mins, 5˜10 cycles; 95° C., 0.5 min, 50° C., 0.5 min, 72° C., 2 mins, 5˜10 cycles; 72° C., 7 mins, 1 cycle.

After the PCR product of the CSK ORF amplified above was separated by an electrophoresis, the PCR product was cloned into pDONR201 or pDONR221 vector (Invitrogen) by a known cloning method to make the CSK ORF clone without the stop codon (Hartley, et al. (2000) DNA cloning using in vitro site-specific recombination. Genome Res. 10, 1788-1795). The completed CSK ORF clone was identified by sequencing.

A clone of SNF1LK (GenBank Acc. No. BCO38504) ORF(open reading frame) clone without a stop codon was purchased from Open Biosystems. SNF1LK ORF clone and CSK ORF clone were cloned into pCMV-DEST-EGFP by a known cloning method to produce CMV-SNF1LK-EGFP and CMV-CSK-EGFP expression vectors (Hartley, et al. (2000) DNA cloning using in vitro site-specific recombination. Genome Res. 10, 1788-1795). The pCMV-DEST-EGFP vector was designed by inserting attR1-ccdB-attR2 sequence into pcDNA3.1/Zeo(+) vector (Invitrogen, Cat. No. V860-20), and then inserting EGFP gene after the attR2 sequence.

After HeLa cells were subcultured to 5,000˜10,000 cells/well on a 96-well plate, the HeLa cells were transfected by the expression vector DNA prepared as above. DNA transfection can be performed by a known method, for example, lipofectamine (purchased from Invitrogen) or Fugene 6 (purchased from Roche). Magnetic materials coated with dasatinib-biotin were into the cells transfected with DNA by the method mentioned in Example 2, and then NDGA (Nordihydroguaiaretic acid, Sigma) was treated to the final concentration of 25˜50 μM. Next, after applying a magnetic field by the method mentioned in Example 2, the cells were observed under a microscope.

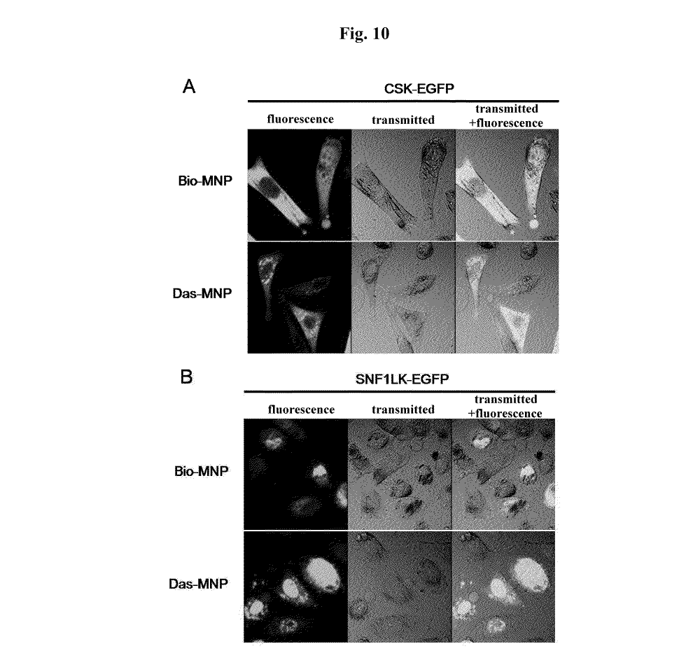

In this embodiment, Olympus fluorescence microscope, FV1000, equipped with the object lens of Uplan Apo 40×/0.85 was used to obtain transmitted images in the cells. As shown in FIG. 10(A), when a surface-modified magnetic material was labeled with a fluorescent material of EGFP protein through a mediator (dasatinib-CSK), the fluorescent pattern of EGFP was identified in a magnetized direction (in a direction of a line of magnetic force). When a biotin-magnetic particle complex (bio-MNP) with no mediator was used as a negative control, however, the fluorescent pattern of EGFP was not identified.

Similarly, as shown in FIG. 10(B), when a surface-modified magnetic material was labeled with a fluorescent material of EGFP protein through a mediator (dasatinib-SNF1LK), the fluorescent pattern of EGFP was identified in a magnetized direction (in a direction of a line of magnetic force). When a biotin-magnetic particle complex (bio-MNP) with no mediator was used as a negative control, however, the fluorescent pattern of EGFP was not identified.

Meanwhile, the fluorescent pattern was appeared and overlapped with the pattern of the magnetic materials observed in a transmitted light image, which means that the fluorescent material was exactly labeled to the surface-modified magnetic material through a mediator of dasatinib-CSK or dasatinib-SNF1LK and thus imaged the magnetized pattern. Therefore, according to the present invention, cellular structures and metabolisms can be easily monitored in a live cell by imaging a pattern of magnetic materials introduced into a live cell in a direction of a line of magnetic force by a label.

In this embodiment, for example, when linkers such as dasatinib and CSK, or dasatinib and SNF1LK are bound with each other to produce a mediator, a magnetic material is labeled with a fluorescent material and its fluorescent pattern was observed. When it is not apparent whether linkers are bound with each other to produce a mediator, labeling of a magnetic material by a fluorescent material can be confirmed by the formation of fluorescent pattern and the co-localization (overlapping) of the magnetized pattern of magnetic materials with the fluorescent pattern. Furthermore, using this method, reactions, roles and signaling pathways of linkers in cellular metabolisms can be monitored.

Although the present invention has been illustrated and described with reference to the exemplified embodiments of the present invention, it should be understood that various changes, modifications and additions to the present invention can be made without departing from the spirit and scope of the present invention.

1-12. (canceled)

13. A method for imaging a pattern of magnetic materials in a live cell comprising:
(a) preparing a plurality of magnetic materials magnetized in a direction of a line of magnetic force by applying a magnetic field, at least one of the magnetic materials being configured into a nanoparticle and the surface of at least one of the magnetic materials being modified;
(b) preparing a mediator comprising a first linker and a second linker which are subjected to identify whether the linkers bind to each other in a live cell;
(c) introducing a plurality of the magnetic materials into each live cell, the first linker being associated with the magnetic material;
(d) providing the live cell with a label capable of imaging a pattern of the magnetic materials in a direction of a line of magnetic force, the second linker being associated with the label;
(e) allowing a bundle of the lines of magnetic force to pass through the live cell in a direction by applying a focused magnetic field to the live cell;
(f) aligning a plurality of the magnetic materials in the live cell with the direction of the line of magnetic force by the applied magnetic field;
(g) identifying a pattern imaged by the label capable of imaging the aligned pattern of the magnetic materials; and
(h) determining that the label has been labeled to the magnetic material in the live cell by binding the second linker to the first linker from the identification of the step (g).

14. The method as claimed in claim 13, further comprising identifying the pattern of the magnetic materials and the pattern imaged by the label, and whether the pattern imaged by the label is co-localized with the pattern of the magnetic materials or not.

15. The method as claimed in claim 13, wherein the magnetic material is a transition metal compound selected from a group consisting of period 4 transition metals such as iron, manganese, chrome, nickel, cobalt, and zinc; their oxides, sulfides, and phosphides; their alloys; and oxides, sulfides, and phosphides of the alloys, or a composition including at least one of them.

16. The method as claimed in claim 15, wherein the magnetic material includes one or mixture of at least two selected from a group consisting of magnetite (Fe₃O₄), maghemite (gamma-Fe₃O₄), cobalt ferrite (CoFe₂O₄), manganese oxide (MnO), manganese ferrite (MnFe₂O₄), iron (Fe)-platinum (Pt) alloy, cobalt (Co)-platinum (Pt) alloy and cobalt (Co).

17. The method as claimed in claim 13, wherein the diameter of the magnetic material is about 1˜1,500 nm.

18. The method as claimed in claim 17, wherein the diameter of the magnetic material is about 20˜350 nm.

19. The method as claimed in claim 13, wherein the saturation magnetization of the magnetic material is above 40 emu (electromagnetic unit)/g.

20. The method as claimed in claim 13, wherein the magnetic material introduced into the live cell is observed as a black dot.

21. The method as claimed in claim 20, wherein the diameter of the black dot is above 300˜1,500 nm.

22. The method as claimed in claim 20, wherein the black dot comprises a single magnetic material or a plurality of magnetic materials locally adjacent to each other.

23. The method as claimed in claim 22, wherein a plurality of black dots exists in the live cell.

24. The method as claimed in claim 13, wherein when the focused magnetic field is applied to the live cell, the magnetic field is applied in a horizontal direction to the bottom on which the live cell is placed.

25. The method as claimed in claim 13, wherein the step of applying the focused magnetic field to the live cell is performed by an apparatus for applying a magnetic field, and the apparatus comprises a cylindrical core consisting of unmagnetized magnetic materials for strengthening the magnetic field and for fixing a container in which the live cell is placed, or a means for increasing the magnetic field gradient provided with a plurality of extensions that support the container.

26-30. (canceled)

31. An apparatus used for imaging a pattern of magnetic materials in a live cell comprising:
a live cell provided with a plurality of magnetic materials magnetized in a direction of a line of magnetic force by applying a magnetic field; at least one of the magnetic materials being configured into a nanoparticle; the surface of at least one of the magnetic materials being modified; and the magnetic material being associated with a first linker of two linkers which are subjected to identify whether the linkers bind to each other in the live cell, and provided with a label capable of imaging a pattern of the magnetic materials, the label being associated with a second linker of two linkers;
a container for receiving and culturing the live cell;
an apparatus for allowing a bundle of the lines of magnetic force to pass through the live cell in a direction by applying a focused magnetic field to the live cell; and
a device for monitoring that the label has been labeled to the magnetic material in the live cell by binding the second linker to the first linker after imaging the pattern of the magnetic materials aligned with the direction of the line of magnetic force and the pattern imaged by the label.

32. The apparatus as claimed in claim 31, wherein the device can identify whether the pattern of the magnetic materials is co-localized with the pattern imaged by the label or not.

33. The apparatus as claimed in claim 31, wherein the apparatus comprises a cylindrical core consisting of unmagnetized magnetic materials for strengthening the magnetic field and for fixing the container in which the live cell is placed, or a means for increasing the magnetic field gradient provided with a plurality of extensions that support the container.
